(19) United States
(12) Patent Application Publication
(10) Pub. No.: US 2022/0301220 A1
(43) Pub. Date: Sep. 22, 2022

(54) METHOD AND DEVICE FOR DISPLAYING TARGET OBJECT, ELECTRONIC DEVICE, AND STORAGE MEDIUM

(71) Applicant: BEIJING SENSETIME TECHNOLOGY DEVELOPMENT CO., LTD., Beijing (CN)

(72) Inventor: Liwei ZHANG, Beijing (CN)

(21) Appl. No.: 17/834,021

(22) Filed: Jun. 7, 2022

Related U.S. Application Data
(63) Continuation of application No. PCT/CN2020/100714, filed on Jul. 7, 2020.

(30) Foreign Application Priority Data
Dec. 19, 2019 (CN) 201911318256.6

(51) Int. Cl. G06T 7/73; A61B 8/00; A61B 8/08

(52) U.S. Cl. CPC G06T 7/73; A61B 8/463; A61B 8/5215; A61B 8/48

(57) ABSTRACT

A method for displaying a target object, an electronic device, and a non-transitory storage medium are provided. The method includes: displaying at least one to-be-analyzed object in response to a first operation for the target object; obtaining an anchor point for determining one of the at least one to-be-analyzed object, in response to a second operation for the target object; determining, according to acquired object distribution images and the anchor point, a range of area where the current to-be-analyzed object corresponding to the anchor point is located in the target object.

### CROSS-REFERENCE TO RELATED APPLICATIONS

This application is a continuation of International Application No. PCT/CN2020/100714, filed on Jul. 7, 2020, which is based on and claims priority to Chinese patent application No. 201911318256.6, filed on Dec. 19, 2019. The contents of International Application No. PCT/CN2020/100714 and Chinese patent application No. 201911318256.6 are hereby incorporated by reference in their entireties.

### BACKGROUND

In two-dimensional (2D) planar display and three-dimensional (3D) model building, for a target object and an anchor point in an operation area (a 2D display area, or a 3D display area obtained by 3D modeling), in order to obtain more clear positional relationships of such as the target object and the anchor point, visual interface design needs to be performed. However, existing interface design cannot clearly show these positional relationships and are not intuitive, causing a user being unable to obtain an accurate judgment result according to the interface design.

### SUMMARY

The disclosure relates to the technical field of displaying visual interfaces, and in particular to a method and device for displaying a target object, an electronic device, and a non-transitory computer-readable storage medium.

According to a first aspect of the embodiments of the present disclosure, provided is a method for displaying a target object, including: displaying at least one to-be-analyzed object in response to a first operation for the target object; obtaining an anchor point for determining one of the at least one to-be-analyzed object, in response to a second operation for the target object; according to acquired object distribution images and the anchor point, determining a range of area where the current to-be-analyzed object corresponding to the anchor point is located in the target object.

In embodiments of the present disclosure, provided is a device for displaying a target object, including: a first response part, configured to: display at least one to-be-analyzed object in response to a first operation for the target object; a second response part, configured to: obtain an anchor point for determining one of the at least one to-be-analyzed object, in response to a second operation for the target object; and an area determination part, configured to determine, according to acquired object distribution images and the anchor point, a range of area where the current to-be-analyzed object corresponding to the anchor point is located in the target object.

In embodiments of the present disclosure, provided is an electronic device, including: a processor; and a memory configured to store processor-executable instructions, wherein the processor is configured to perform following operations: displaying at least one to-be-analyzed object in response to a first operation for the target object; obtaining an anchor point for determining one of the at least one to-be-analyzed object, in response to a second operation for the target object; according to acquired object distribution images and the anchor point, determining a range of area where the current to-be-analyzed object corresponding to the anchor point is located in the target object.

In embodiments of the present disclosure, provided is a non-transitory computer-readable storage medium having stored thereon computer program instructions that, when executed by a processor, implement a method for displaying a target object, the method including: displaying at least one to-be-analyzed object in response to a first operation for the target object; obtaining an anchor point for determining one of the at least one to-be-analyzed object, in response to a second operation for the target object; according to acquired object distribution images and the anchor point, determining a range of area where the current to-be-analyzed object corresponding to the anchor point is located in the target object.

In embodiments of the present disclosure, provided is a computer program including computer-readable code that, when running in an electronic device, causes a processor in the electronic device to execute the method for displaying a target object in one or more of the embodiments described above.

It is to be understood that the foregoing general description and the following detailed description are both exemplary and explanatory only and are not restrictive of the disclosure.

Other features and aspects of the disclosed embodiments will become apparent from the following detailed description of exemplary embodiments with reference to the accompanying drawings.

### BRIEF DESCRIPTION OF THE DRAWINGS

The accompanying drawings, which are incorporated in and constitute a part of this specification, illustrate embodiments consistent with the disclosure and together with the description serve to describe the technical solutions of the disclosure.

FIG. 1 illustrates a flowchart of a method for displaying a target object according to embodiments of the disclosure.

FIG. 2 illustrates a schematic diagram of object identification legends for a target object which is a blood vessel according to embodiments of the disclosure.

FIG. 3 illustrates a schematic diagram of positional relationships of a target object which is a blood vessel, and a corresponding anchor point according to embodiments of the disclosure.

FIG. 4 illustrates a block diagram of a device for displaying a target object according to embodiments of the disclosure.

FIG. 5 illustrates a block diagram of an electronic device according to embodiments of the disclosure.

FIG. 6 illustrates a block diagram of an electronic device according to embodiments of the disclosure.

### DETAILED DESCRIPTION

Various exemplary embodiments, features, and aspects of the present disclosure will be described in detail below with reference to the accompanying drawings. The same reference numerals in the figures indicate identical or similar elements. Although various aspects of the embodiments are illustrated in the drawings, the drawings are not necessarily drawn to scale unless otherwise indicated.

The special term “exemplary” here means “serving as an example, embodiment, or illustration.” Any embodiment described herein as “exemplary” may not be construed as being superior or better than other embodiments.

The term “and/or” as used herein merely describes an association relationship of associated objects, and means that there may be three relationships. For example, A and/or B may represent three situations: independent existence of A, existence of both A and B, and independent existence of B. Additionally, the term “at least one” as used herein denotes any one of multiple, or any combination of at least two of the multiple. For example, including at least one of A, B and C may denote the inclusion of any one or more elements selected from the group consisting of A, B, and C.

In addition, for describing the present disclosure better, many details are provided in the implementations below. It is to be appreciated by those skilled in the art that the present disclosure may also be practiced without certain details. In some embodiments, methods, means, elements, and circuits well known to those skilled in the art are not been described in detail, to highlight the subject of the disclosure.

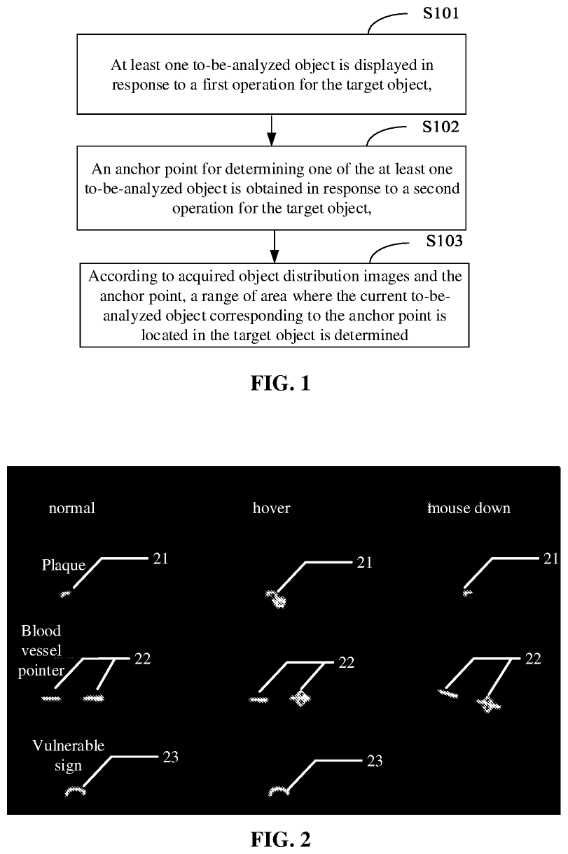

FIG. 1 illustrates a flowchart of a method for displaying a target object according to embodiments of the present disclosure. The method is applied to a device for displaying a target object. For example, in the case where the device is deployed for execution in a terminal device, a server or other processing devices, at least one to-be-analyzed object (such as a nidus in a lesion region) may be displayed and positioned, and a range of area where the to-be-analyzed object is distributed may be determined. The terminal device may be user equipment (UE), a mobile device, a cellular telephone, a cordless telephone, a personal digital assistant (PDA), a handheld device, a computing device, a vehicle-mounted device, a wearable device, or the like. In some possible implementations, the processing method may be implemented by a processor invoking computer-readable instructions stored in a memory. As illustrated in FIG. 1, the procedure includes the following actions S101 to S103.

At S101, at least one to-be-analyzed object is displayed in response to a first operation for the target object.

In some possible implementations, the target object is a blood vessel as an example. The first operation may be an operation of selecting the blood vessel, and the at least one to-be-analyzed object may be a vascular plaque in a lesion region, or a nidus in another non-vascular region. When the blood vessel is selected, a vascular plaque in at least one lesion region in the blood vessel, and/or a nidus in at least one non-vascular region may be displayed.

At S102, an anchor point for determining one of the at least one to-be-analyzed object is obtained, in response to a second operation for the target object.

In some possible implementations, the at least one to-be-analyzed object may be a vascular plaque in a lesion region, or a nidus in another non-vascular region. With the target object being a blood vessel as an example, the at least one to-be-analyzed object may be multiple vascular plaques, and the second operation may be an operation of positioning any one of the multiple vascular plaques. The at least one to-be-analyzed object may also be multiple nidi in a non-vascular region, and the second operation may be an operation of positioning any of the nidi in the non-vascular region. By parsing the second operation, an anchor point corresponding to the second operation may be obtained, and the anchor point may be used to position any of the at least one to-be-analyzed object.

At S103, according to acquired object distribution images and the anchor point, a range of area where the to-be-analyzed object corresponding to the anchor point is located in the target object is determined.

In some possible implementations, the object distribution images may include: an image of a distribution range of the at least one to-be-analyzed object in the target object, for example, multiple cross-sectional views of the blood vessel corresponding to positions on the blood vessel. Here, different cross-sectional views of the blood vessel are obtained at different regional positions on the blood vessel.

By means of the embodiments of the present disclosure, object distribution images can be obtained. According to the distribution range of at least one to-be-analyzed object in the target object in the object distribution images, an intuitive interface design can assist the user to obtain an accurate judgment result of the object distribution range.

In an example, in some possible implementations, before displaying the at least one to-be-analyzed object in response to the first operation for the target object, the method may further include the following actions: obtaining a feature vector corresponding to the at least one to-be-analyzed object; each of the at least one to-be-analyzed object is recognized according to the feature vector and a recognition network; and each of the at least one to-be-analyzed object is identified to obtain a display identifier. The at least one to-be-analyzed object may include: multiple objects displayed according to display identifiers.

By means of the embodiments of the present disclosure, the at least one to-be-analyzed object can be recognized according to the feature vectors and the recognition network, and each of the at least one to-be-analyzed object can be identified to obtain a display identifier. By displaying the at least one to-be-analyzed object through the display identifier, the user can be assisted to quickly determine the to-be-analyzed object according to the intuitive interface design, and perform needed analysis judgment on the to-be-analyzed object.

In some possible implementations, after determining the range of area where the current to-be-analyzed object corresponding to the anchor point is located in the target object, the method may further include: in response to a third operation (an operation of selecting a vascular plaque) for the current to-be-analyzed object corresponding to the anchor point, a feature object (such as a vulnerable sign under the vascular plaque) that corresponds to the current to-be-analyzed object corresponding to the anchor point is displayed. The feature object has a nature of lesion different from that of the current to-be-analyzed object corresponding to the anchor point.

In the embodiments of the present disclosure, at least one to-be-analyzed object (such as a vascular plaque in a lesion region) is displayed in response to a first operation for the target object; an anchor point for determining one of the at least one to-be-analyzed object is obtained in response to a second operation for the target object; according to acquired object distribution images (such as cross sections of the blood vessel corresponding to the anchor point of the vascular plaque) and the anchor point, the range of area where the current to-be-analyzed object corresponding to the anchor point is located in the target object is determined. By means of the embodiments of the present disclosure, positional relationships of such as the target object and the anchor point can be clearly obtained in the visual interface design, and the display effect of the interface design is intuitive, so that the user can obtain accurate judgment results based on the intuitive interface design.

Embodiments of the present disclosure are described below by way of example. Firstly, the action S101 that at least one to-be-analyzed object is displayed in response to a first operation for the target object is explained. In this embodiment, the target object is a blood vessel and the to-be-analyzed object is a vascular plaque as an example. When a first operation for the blood vessel (such as an operation of selecting the blood vessel) is received, at least one vascular plaque in a lesion region in the blood vessel is displayed. Before action S101, a feature vector corresponding to the at least one vascular plaque in the blood vessel may be obtained, and each of the at least one vascular plaque is recognized according to the feature vector and a recognition network. In a possible implementation, a display identifier may be added to each of the at least one vascular plaque, and after receiving the first operation for the blood vessel, each vascular plaque is displayed according to the display identifier of the vascular plaque.

Following the above explanation of action S101, it is continued to explain action S102 that an anchor point for determining one of the at least one to-be-analyzed object is obtained in response to a second operation for the target object. In this embodiment, after the blood vessel is displayed and the at least one vascular plaque is displayed, an anchor point of a vascular plaque corresponding to a second operation can be obtained after receiving the second operation for the blood vessel (such as an operation of positioning any vascular plaque displayed in the blood vessel).

Following the above explanation of action S102, it is continued to explain action S103 that a range of area where the to-be-analyzed object corresponding to the anchor point is located in the target object is determined according to acquired object distribution images and the anchor point. In this embodiment, object distribution images corresponding to the blood vessel are acquired. The object distribution images may include cross-sectional views of the blood vessel at different positions of the blood vessel. According to the cross-sectional views of the blood vessel at the different positions of the blood vessel and the anchor point, a range of area where the vascular plaque selected in action S102 is located in the blood vessel can be obtained.

Following the above explanation of action S103, some other possible embodiments are described. After determining the range of area where the vascular plaque selected in action S102 is located in the blood vessel, a third operation for the vascular plaque (for example, an operation of selecting the vascular plaque) may be acquired, and a feature object corresponding to the vascular plaque may be displayed. For example, a vulnerable sign corresponding to the vascular plaque may be displayed. In some possible implementations, the vulnerable sign may have a nature of lesion different from that of the current to-be-analyzed object.

By means of the embodiments of the present disclosure, the displayed feature object can be obtained in response to the third operation, and an object having a nature of lesion different from that of the to-be-analyzed object can be obtained through the feature object.

FIG. 2 illustrates a schematic diagram of object identification legends for a target object being a blood vessel according to embodiments of the present disclosure, including: a display identifier 21 of a plaque on the blood vessel, a display identifier of a vulnerable sign 23 corresponding to the plaque, a display identifier 22 of a blood vessel pointer for positioning, and the like. The embodiments of the present disclosure are not limited to the object identification legends illustrated in FIG. 2. Any identification forms that can distinguish different objects from one another, and are capable of ensuring that multiple object legends are highly distinguished from each other shall fall within the scope of the embodiments of the present disclosure.

As illustrated in FIG. 2, at least one to-be-analyzed object can be recognized in the target object according to the artificial intelligence technology by such as the feature vectors and the recognition network described above. Correspondingly, the at least one to-be-analyzed object is identified and displayed to the user by the object identification legends in FIG. 2 respectively. In some possible implementations, the object identification legends may include, but are not limited to, plaques, blood vessel pointers, and vulnerable signs, in the case where the target object is a blood vessel. Different object legends are highly distinguished from each other. Thus, lesions of different natures are displayed with highly distinguishable display identifiers, which is convenient for a user to review; furthermore, positional relationships of such as the target object and the anchor point corresponding to the display identifiers can be clearly obtained through different display identifiers in the visual interface design, so that the user can obtain an accurate judgment results of the lesion according to the visual interface design.

In some possible implementations, with the target object being a blood vessel as an example, the to-be-analyzed object may be a vascular plaque in a lesion region displayed in response to a first operation.

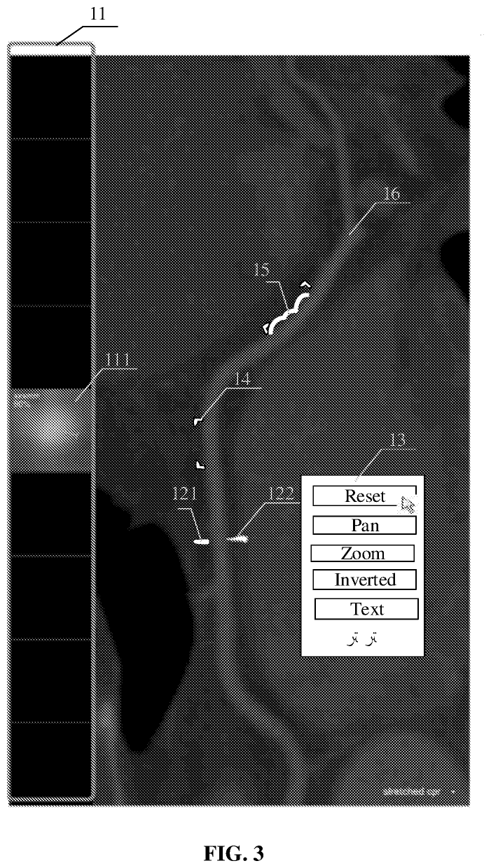

FIG. 3 illustrates a schematic diagram of positional relationships of a target object which is a blood vessel, and an anchor point on according to embodiments of the present disclosure.

As illustrated FIG. 3, the object distribution images 11 may be multiple cross-sectional views of the blood vessel corresponding to positions on the blood vessel. An anchor point for determining any of the at least one to-be-analyzed object, i.e., the vascular plaque is obtained, such as an anchor point limited by the first position identifier 121 and the second position identifier 122 in response to a second operation (which may be an operation of positioning any one of multiple vascular plaques in the blood vessel) for the target object (i.e., the blood vessel).

According to the acquired object distribution images, multiple cross-sectional views of the blood vessel corresponding to positions on the blood vessel are acquired. According to the position of the anchor point limited by the first position identifier 121 and the second position identifier 122 in the multiple cross-sectional views of the blood vessel, a range of section in which the to-be-analyzed object corresponding to the anchor point is located in the target object is determined. Thus, the user can learn that the plaque corresponding to the present anchor point is positionally located in a certain range of section in the entire blood vessel (for example, the screen shot 111 illustrated distinguishably from those at other positions among the object distribution images 11).

FIG. 3 also includes positional relationships of to-be-analyzed objects and anchor points, and an operation menu 13 triggered by a right button of a mouse in the case where the target object is a blood vessel.

The to-be-analyzed objects not only include a vascular plaque 14, but may also include vulnerable signs 15 located under the vascular plaque 14 of the blood vessel 16. It may be triggered to display the vulnerable signs after a vascular plaque is selected.

In some possible implementations, a clear and intuitive interface display effect can be obtained by the different display modes and the displayed position relationship of the to-be-analyzed objects, thereby facilitating a user in viewing and determine the positional relationship of to-be-analyzed objects. For example, the blood vessel may be selected according to the first operation of the user, to display all plaques under the blood vessel on the interface. Alternatively, all vascular plaques under the blood vessel may be directly displayed according to actual application requirements, without being limited to being triggered by an operation. Any one of the vascular plaques is selected for view, according to the second operation of the user. The present position is obtained according to the anchor point and the multiple cross-sectional views of blood vessel. That is, the position of the vascular plaque, corresponding to the anchor point pointed to by the mouse pointer, in the area of the entire blood vessel is determined according to the position of the anchor point in the multiple cross-sectional views of the blood vessel. Further, the vascular plaque may also be selected to display the location and range of vulnerable signs under the vascular plaque.

In some possible implementations, corresponding operations may be performed by directly clicking on the operation menu 13. There is no need to perform an additional switch action to enter a next operation, thereby simplifying the user operations and increasing the speed in interaction and feedback.

In some possible implementations, the display of the operation menu 13 may be triggered by right-click. The operation menu includes, but is not limited to, a reset option, a pan option, a zoom option, an inverted option, and a text option. By further selecting a target option in the operation menu 13, the operation corresponding to the target option can be selected. For example, after the user selects the pan option in the operation menu 13, the operation corresponding to the panning option is switched to. Namely, the pan operation is switched to.

In summary, with the disclosed embodiments, with different interactive display corresponding to different user operations, multiple lesions (such as vascular plaques, and vulnerable signs) of different natures can be distinguished and displayed. The position of the presently positioned vascular plaque in the range of the entire blood vessel can be learned based on the anchor point corresponding the present plaque and the above multiple cross-sectional views. Therefore, better positioning can be achieved based on the interface display identifiers and the interactive display.

In some possible implementations, the action that the range of area where the current to-be-analyzed object corresponding to the anchor point is located in the target object is determined according to the acquired object distribution images and the anchor point may include: a reference image corresponding to the anchor point is obtained from the object distribution images; and the range of area where the current to-be-analyzed object corresponding to the anchor point is located in the target object is determined according to a serial number or ranking position of the reference image among the object distribution images. For example, if the serial number is 2, the ranking position is the second in the multiple cross sections, indicating that the range of area is at an upper position compared to the initial anchor point of the target object (e.g., the initial anchor point is in the middle of the target object).

By means of the embodiments of the present disclosure, a reference image corresponding to the anchor point can be obtained from the object distribution images, so that the range of area where the to-be-analyzed object corresponding to the anchor point is located in the target object can be determined according to a serial number or ranking position of the reference image among the object distribution images.

In some possible implementations, after the reference image corresponding to the anchor point is obtained from the object distribution images, the method may further include: the reference image corresponding to the anchor point is displayed in a display mode different from a mode of displaying a non-reference image among the object distribution images to distinguish the reference image from the non-reference image, and an obtained display result is fed back to a user in real time. For example, among nine cross-sectional views corresponding to the positions on the blood vessels, the cross-sectional view corresponding to the present anchor point may be highlighted differently from the other cross-sectional views. Therefore, according to the anchor point and the highlight, the user can learn which range of section the plaque corresponding to the present anchor point is located in the entire blood vessel, thereby achieving better positioning and facilitating real-time viewing by the user.

By means of the embodiments of the present disclosure, the reference image corresponding to the anchor point and a non-reference image among the object distribution images may be distinguished from each other by displaying them in different display modes respectively. For example, the reference image may be highlighted to distinguish from a non-reference image, so as to assist the user to quickly obtain the reference image according to the intuitive interface design, so as to perform the needed analysis and judgment on the to-be-analyzed object.

In some possible implementations, the method may further include: in response to a position change of the anchor point, a range of area where a position-changed to-be-analyzed object is located in the target object is updated to obtain an updated result, that is, a new range of area different from that displayed in a previous area, by switching the current to-be-analyzed object to the position-changed to-be-analyzed object and synchronizing the position change of the anchor point to the object distribution images. For example, the vascular plaque may be switched along with an anchor point and synchronized to a corresponding cross-section view among the multiple cross-sectional views, so as to feed a new range of area of the vascular plaque corresponding to the position updated and changed anchor point in the entire blood vessel back to the user in real time, for easy view by the user.

By means of the embodiments of the present disclosure, in response to the position change of the anchor point, the range of area where the to-be-analyzed object corresponding to the position-changed anchor point is located in the target object can be synchronously updated in real time, to assist the user to switch to, in real time, the updated result obtained after the synchronous update, so as to make the required analysis and judgment on the to-be-analyzed object.

Hereinafter, an exemplary application of the embodiments of the disclosure in an actual application scenario will be described.

In medical images of hearts or some multi-level presentation, there may be such as blood vessels, plaques and vulnerable signs. Images of stenosis and plaque positions, and positional relationships related with the degree of stenosis need to be viewed when viewing blood vessels. Image of cross sections corresponding to the blood vessel also need to be viewed. Artificial intelligence is generally not used in existing technologies, and it is unable to automatically recognize lesion regions and lesion locations on all blood vessels, no specific nature or identifier is indicated, and no cross-sectional view corresponding to a position on the blood vessel is reflected, thus being unable to clearly reflect positional relationships of the lesions with the range of the blood vessels.

By means of embodiments of the present disclosure, a range of a plaque on a blood vessel and a range of a vulnerable sign on the blood vessel can be intuitively presented, and a distinguishable and intuitive mode of interaction for example plaque switching can be supported. It is also possible to indicate, on the blood vessel, the range of the cross-sectional views of a region corresponding to a pointer, so as to facilitate judgment based on the image.

As illustrated in FIG. 3, a curved planar reconstruction (CPR) image needs to be referred to when viewing a blood vessel. The number of plaques, the range and position of a plaque in the blood vessel, as well as the position and range of a vulnerable sign can be seen in the image, so that the physician can make judgment and positioning based on the entire artificial intelligence (AI) result clearly and visually.

When moving the blood vessel pointer to view the image of a corresponding cross section, i.e., in the zone 11 in FIG. 3, the range relationship of the plaque in the nine cross sections on the blood vessel can be seen in real time, facilitating positioning for the physician.

When diagnosing a cardiovascular disease, the physician needs to confirm and analyze conclusions given in the image and corresponding lesion regions. At this time, the physician needs to review and confirm blood vessels one by one. Reference should be made to the CPR image when viewing the blood vessels. The number of plaques, and the range and location of a plaque in the blood vessel, as well as the location and range of a vulnerable sign in the blood vessel can be seen in the CPR image, and the plaques and vulnerable signs can be switched directly on the image and synchronized in the list. This is convenient for the physician to make judgment and positioning based on the entire AI result clearly and intuitively.

When the blood vessel pointer is moved to view corresponding cross-sectional images, nine corresponding cross-sectional views may be presented in real time in FIG. 3. Moreover, while moving the blood vessel pointer, the range of area of a plaque in the cross sections of the blood vessel is displayed synchronously, so that the range relationship is clearly reflected and the positioning is better realized for the physician.

Embodiments of the present disclosure may be applied to an image reading system in an imaging department; scanning stations such as computed tomography (CT), magnetic resonance (MR), and positron emission tomography (PET); and all logical operations having a correspondence relationship, such as AI-assisted diagnosis, an AI labeling system, telemedicine diagnosis, and cloud platform-assisted intelligent diagnosis.

It may be appreciated by those skilled in the art that in the above method of embodiments, the order in which the actions are written does not imply a strict order of execution to constitute any limitation on the implementation process, and that the order in which the actions are executed should be determined in terms of their functions and possible internal logic.

The above-mentioned method embodiments provided in the present disclosure may be combined with each other to form a combined embodiment without departing from the principle and logics, and which will not be described here in detail.

In addition, the present disclosure also provides a device for displaying a target object, an electronic device, a computer-readable storage medium, and a program that can all be used to implement any method for displaying a target object provided in the present disclosure. The corresponding technical solutions and description may refer to the corresponding content in the method part, and will not be described again.

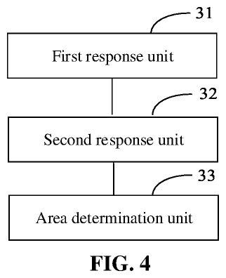

FIG. 4 illustrates a block diagram of a device for displaying a target object according to embodiments of the present disclosure. As illustrated in FIG. 4, the device for displaying the target object includes a first response part 31, a second response part 32 and an area determination part 33. The first response part 31 is configured to: display at least one to-be-analyzed object in response to a first operation for the target object. The second response part 32 is configured to: obtain an anchor point for determining one of the at least one to-be-analyzed object, in response to a second operation for the target object. The area determination part 33 is configured to: determine, according to acquired object distribution images and the anchor point, a range of area where the current to-be-analyzed object corresponding to the anchor point is located in the target object.

In this embodiment and other embodiments, “part” may be part of a circuit, part of a processor, part of a program or software, etc., of course may be a unit, or may be a module or non-modular.

In a possible implementation, the device may further include a third response part. The third response part is configured to: display a feature object that corresponds to the current to-be-analyzed object corresponding to the anchor point in response to a third operation for the current to-be-analyzed object corresponding to the anchor point. The feature object has a nature of lesion different from that of the current to-be-analyzed object corresponding to the anchor point.

In a possible implementation, the object distribution images may include: an image of a distribution range of the at least one to-be-analyzed object in the target object.

In a possible implementation, the area determination part is configured to: obtain, from the object distribution images, a reference image corresponding to the anchor point; and determine, according to a serial number or ranking position of the reference image among the object distribution images, the range of area where the current to-be-analyzed object corresponding to the anchor point is located in the target object.

In a possible implementation, the device may further include a feedback part. The feedback part is configured to: display the reference image corresponding to the anchor point in a display mode different from a mode of displaying a non-reference image among the object distribution images, to distinguish the reference image from the non-reference image; and feed an obtained display result back to a user in real time.

In a possible implementation, the device may further include an area update part. The area update part is configured to: in response to a position change of the anchor point, update a range of area where a position-changed to-be-analyzed object is located in the target object to obtain an updated result by switching the current to-be-analyzed object to the position-changed to-be-analyzed object and synchronizing the position change of the anchor point to the object distribution images.

In a possible implementation, the device may further include an object identification part. The object identification part is configured to: obtain a feature vector corresponding to the at least one to-be-analyzed object; recognize each of the at least one to-be-analyzed object according to the feature vector and a recognition network; and identify each of the at least one to-be-analyzed object to obtain a display identifier. Each of the at least one to-be-analyzed object is displayed according to the display identifier.

In some embodiments, the device provided in the embodiments of the present disclosure may have functions or include parts that may be configured to perform the methods described in the above method embodiments, the implementation of which may refer to the description of the above method embodiments and will not be described herein for brevity.

In this embodiment and other embodiments of the present disclosure, “part” may be part of a circuit, part of a processor, part of a program or software, etc., of course may be a unit, or may be a module or non-modular.

In embodiments of the present disclosure, also provided is a computer-readable storage medium having stored thereon computer program instructions that, when executed by a processor, implement the method described above. The computer-readable storage medium may be a volatile computer-readable storage medium or a non-volatile computer-readable storage medium.

In embodiments of the present disclosure, also provided is a computer program product including computer-readable code that, when running in a device, causes a processor in the device to execute instructions for implementing a method for displaying a target object as provided in any of the above embodiments.

In embodiments of the present disclosure, also provided is another computer program product for storing computer-readable instructions that, when executed, cause a computer to perform operations of the method for displaying a target object provided in any of the above embodiments.

In the embodiments of the present disclosure, at least one to-be-analyzed object (such as a vascular plaque in a lesion region) is displayed in response to a first operation for the target object; an anchor point for determining one of the at least one to-be-analyzed object is obtained, in response to a second operation for the target object; according to acquired object distribution images (such as cross-sections of the blood vessel corresponding to the anchor point of the vascular plaque) and the anchor point, a range of area where the current to-be-analyzed object corresponding to the anchor point is located in the target object is determined. By means of the embodiments of the present disclosure, positional relationships of such as the target object and the anchor point can be clearly obtained in the visual interface design, and the display effect of the interface design is intuitive, so that the user can obtain accurate judgment results based on the intuitive interface design.

The computer program product may be implemented in hardware, software, or a combination thereof. In an alternative embodiment, the computer program product is embodied as a computer storage medium, and in another alternative embodiment, the computer program product is embodied as a software product, such as a Software Development Kit (SDK).

Embodiments of the present disclosure further provide an electronic device including a processor; and a memory configured to store processor-executable instructions. The processor is configured to invoke the instructions stored in the memory to perform the above method.

The electronic device may be provided as a terminal, a server, or other forms of device.

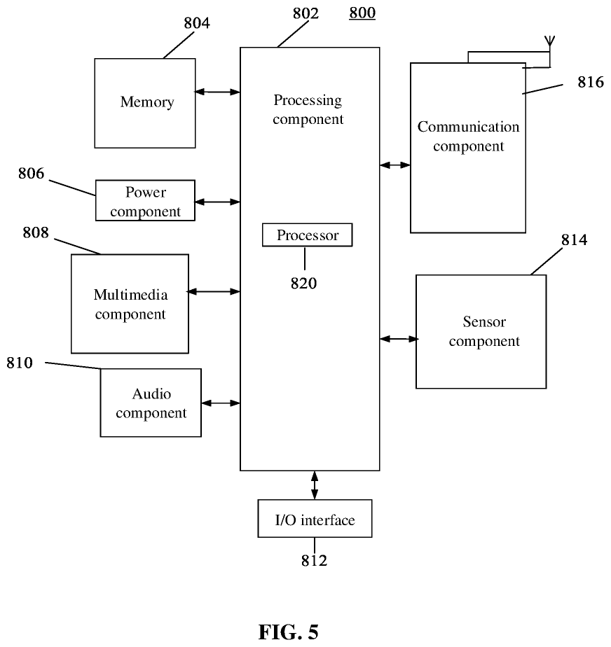

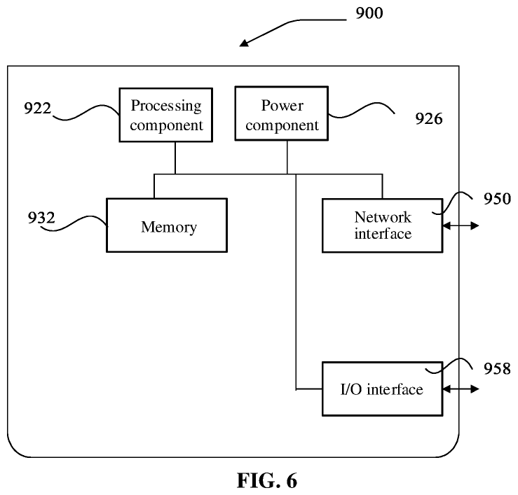

FIG. 5 illustrates a block diagram of an electronic device 800 according to an exemplary embodiment. For example, the electronic device 800 may be a mobile phone, a computer, a digital broadcast terminal, a messaging device, a game console, a tablet device, a medical device, an exercise device, a personal digital assistant, or the like.

Referring to FIG. 5, the electronic device 800 may include one or more of a processing component 802, a memory 804, a power component 806, a multimedia component 808, an audio component 810, an input/output (I/O) interface 812, a sensor component 814, and a communication component 816.

The processing component 802 generally controls the overall operation of the electronic device 800, such as operations associated with displays, telephone calls, data communications, camera operations, and recording operations. The processing component 802 may include one or more processors 820 to execute instructions to perform all or some of the actions of the methods described above. In addition, the processing component 802 may include one or more modules to facilitate interaction between processing component 802 and other components. For example, the processing component 802 may include a multimedia module to facilitate interaction between the multimedia component 808 and the processing component 802.

The memory 804 is configured to store various types of data to support operation at electronic device 800. Examples of such data include instructions of any application or method configured to operate on electronic device 800, contact data, phone book data, messages, pictures, video, etc. The memory 804 may be implemented by any type of volatile or non-volatile storage device or a combination thereof, such as a Static Random-Access Memory (SRAM), an Electrically Erasable Programmable read only memory (EEPROM), an Erasable Programmable Read-Only Memory (EPROM), a Programmable Read-Only Memory (PROM), a Read-Only Memory (ROM), a magnetic memory, a flash memory, a magnetic disk, or an optical disk.

The power component 806 provides power to various components of electronic device 800. Power component 806 may include a power management system, one or more power supplies, and other components associated with generating, managing, and distributing power for electronic device 800.

The multimedia component 808 includes a screen providing an output interface between the electronic device 800 and a user. In some embodiments, the screen may include a Liquid Crystal Display (LCD) and a Touch panel (TP). If the screen includes a touch panel, the screen may be implemented as a touch screen to receive input signals from a user. The touch panel includes one or more touch sensors to sense gestures on the touch, slide, and touch panel. The touch sensor may not only sense the boundary of a touch or slide action, but also detect the duration and pressure associated with the touch or slide action. In some embodiments, the multimedia component 808 includes a front-facing camera and/or a rear-facing camera. When the electronic device 800 is in an operation mode, such as a shooting mode or a video mode, the front-facing camera and/or the rear-facing camera may receive external multimedia data. Each of the front and rear cameras may be a fixed optical lens system or have a focal length and optical zoom capability.

The audio component 810 is configured to output and/or input audio signals. For example, the audio component 810 includes a microphone (MIC) configured to receive an external audio signal when the electronic device 800 is in an operating mode, such as a call mode, a recording mode, and a speech recognition mode. The received audio signal may be further stored in memory 804 or transmitted via communication component 816. In some embodiments, the audio component 810 further includes a speaker for outputting an audio signal.

The I/O interface 812 provides an interface between the processing component 802 and a peripheral interface module, which may be a keyboard, a click wheel, a button, or the like. These buttons may include, but are not limited to, a homepage button, a volume button, a start button, and a lock button.

The sensor component 814 includes one or more sensors configured to provide state evaluation of various aspects of the electronic device 800. For example, the sensor component 814 may detect an on/off state of the electronic device 800, a relative positioning of the components, such as a display and keypad of the electronic device 800. The sensor component 814 may also detect a change in position of the electronic device 800 or one of the components of the electronic device 800, the presence or absence of user contact with the electronic device 800, an orientation or acceleration/deceleration of the electronic device 800, and a change in temperature of the electronic device 800. The sensor component 814 may include a proximity sensor configured to detect the presence of a nearby object without any physical contact. The sensor component 814 may also include a photosensor, such as a Complementary Metal Oxide Semiconductor (CMOS) or Charge-coupled Device (CCD) image sensor, configured for use in imaging applications. In some embodiments, the sensor component 814 may also include an acceleration sensor, a gyroscope sensor, a magnetic sensor, a pressure sensor, or a temperature sensor.

The communication component 816 is configured to facilitate wired or wireless communication between the electronic device 800 and other devices. The electronic device 800 may access a wireless network based on a communication standard, such as wireless fidelity (Wi-Fi), 2^{nd}-Generation wireless telephone technology (2G) or 3^{rd}-Generation wireless telephone technology (3G), or a combination thereof. In one exemplary embodiment, communication component 816 receives broadcast signals or broadcast-related information from an external broadcast management system via a broadcast channel. In one exemplary embodiment, the communication component 816 further includes a Near Field Communication (NFC) part to facilitate short-range communication. For example, the NFC part may be implemented based on Radio Frequency Identification (RFID) technology, Infrared Data Association (IrDA) technology, Ultra Wide Band (UWB) technology, Bluetooth (BT) technology, or other technologies.

In an exemplary embodiment, the electronic device 800 may be implemented by one or more Application-Specific Integrated Circuits (ASICs), Digital Signal Processors (DSPs), Digital signal processing devices (DSPDs), programmable logic devices (PLDs), Field Programmable Gate Arrays (FPGAs), controllers, microcontrollers, microprocessors, or other electronic components for performing the above methods.

In an exemplary embodiment, a non-volatile computer-readable storage medium is also provided, such as a memory 804 containing computer program instructions executable by a processor 820 of the electronic device 800 to perform the methods described above.

FIG. 6 illustrates a block diagram of an electronic device 900 according to an exemplary embodiment. For example, electronic device 900 may be provided as a server. Referring to FIG. 6, electronic device 900 includes processing component 922, which further includes one or more processors; and memory resources represented by a memory 932, for storing instructions, such as an application, that may be executed by the processing component 922. The application stored in memory 932 may include one or more modules each corresponding to a set of instructions. In addition, the processing component 922 is configured to execute instructions to perform the methods described above.

The electronic device 900 may also include a power component 926 configured to perform power management of the electronic device 900, a wired or wireless network interface 950 configured to connect the electronic device 900 to a network, and an input/output (I/O) interface 958. The electronic device 900 may operate based on an operating system stored in memory 932, such as Windows Server™, Mac OS XTM, Unix™, Linux™, FreeBSD™, or the like.

In an exemplary embodiment, a non-volatile computer-readable storage medium is also provided, such as a memory 932 including computer program instructions executable by a processing component 922 of the electronic device 900 to perform the methods described above.

Accordingly, in embodiments of the present disclosure, also provided is a computer program including computer-readable code that, when running in an electronic device, causes a processor in the electronic device to execute the method for implementing the method for displaying a target object as provided in any of the above embodiments.

Embodiments of the present disclosure may be systems, methods, and/or computer program products. A computer program product may include a computer-readable storage medium having stored thereon computer-readable program instructions that, when executed by a processor, implement various aspects of embodiments of the present disclosure.

The computer-readable storage medium may be a tangible device that may hold and store instructions for use by the instruction execution device. The computer-readable storage medium may be, for example, but not limited to, an electrical storage device, a magnetic storage device, an optical storage device, an electromagnetic storage device, a semiconductor storage device, or any suitable combination thereof. Examples (non-exhaustive list) of computer-readable storage media include a portable computer disk, a hard disk, a Random Access Memory (RAM), a Read Only Memory (ROM), an Erasable Programmable Read-Only Memory (EPROM) (or a flash memory) or a flash memory, a Static Random Access Memories (SRAM), a Compact Disc Read-Only Memory (CD-ROMs), a Digital Video Discs (DVD), a memory stick, a floppy disk, a mechanical encoding device e.g., a punched card or an in-slot raised structures with instructions stored therein, or any suitable combination thereof. As used herein, the computer-readable storage medium is not to be construed as an instantaneous signal itself, such as radio waves or other freely propagating electromagnetic waves, electromagnetic waves propagating through a waveguide or other transmission medium (e.g., an optical pulse through a fiber optic cable), or an electrical signal transmitted through a wire.

The computer-readable program instructions described herein may be downloaded to an external computer or external storage device from a computer-readable storage medium to various computing/processing devices, or via a network such as the Internet, a local area network, a wide area network, and/or a wireless network. The network may include copper transmission cables, fiber optic transmission, wireless transmission, routers, firewalls, switches, gateway computers, and/or edge servers. A network adapter card or network interface in each computing/processing device receives computer-readable program instructions from the network and forwards the computer-readable program instructions for storage in the computer-readable storage medium in the respective computing/processing device.

The computer program instructions used to perform the operations of the present disclosure may be assembly instructions, Industry Standard Architecture (ISA) instructions, machine instructions, machine-related instructions, microcode, firmware instructions, state setting data, or source or object code written in any combination of one or more programming languages, including object-oriented programming languages such as Smalltalk and C++, and conventional procedural programming languages such as “C” language or similar programming languages. The computer-readable program instructions may be executed entirely on the user computer, or partly on the user computer, or as a separate software package, or partly on the user computer and partly on the remote computer, or entirely on the remote computer or server. In the case involving a remote computer, the remote computer may be connected to the user computer through any kind of network including a local area network (LAN) or a Wide Area Network (WAN), or may be connected to an external computer (e.g., connected through the Internet using an Internet service provider). In some embodiments, various aspects of the present disclosure are implemented by personalizing an electronic circuit, such as a programmable logic circuit, a Field Programmable Gate Array (FPGA), or a Programmable Logic Array (PLA), with the status information of the computer-readable program instructions.

Various aspects of the present disclosure are described herein with reference to flow charts and/or block diagrams of methods, device (systems), and computer program products in accordance with embodiments of the present disclosure. It should be understood that each block in the flowcharts and/or block diagrams, and combinations of blocks in the flowcharts and/or block diagrams may be implemented by computer readable program instructions.

The computer-readable program instructions may be provided to a processor of a general purpose computer, special purpose computer, or other programmable data processing device to produce a machine such that the instructions, when executed by the processor of the computer or other programmable data processing device, produce means for implementing the functions/acts specified in one or more blocks in the flowchart and/or block diagram. The computer-readable program instructions may also be stored in a computer-readable storage medium that cause a computer, programmable data processing device, and/or other devices to operate in a particular manner, such that the computer-readable medium having the instructions stored thereon includes an article of manufacture that includes instructions to implement various aspects of the functions/acts specified in one or more blocks in the flowchart and/or block diagram.

Computer-readable program instructions may also be loaded onto a computer, other programmable data processing devices, or other devices such that a series of operational blocks are performed on the computer, other programmable data processing devices, or other devices to produce a computer-implemented process such that the instructions that are executed on the computer, other programmable data processing devices, or other devices implement the functions/actions specified in one or more blocks in the flowcharts and/or block diagrams.

The flowcharts and block diagrams in the drawings illustrate architectures, functions, and operations that may be realized for the systems, methods, and computer program products in accordance with various embodiments of the present disclosure. In this regard, each block in a flowchart or block diagram may represent a module, a program segment, or part of instructions that contain one or more executable instructions for implementing a specified logical function. In some alternative implementations, the functions noted in the blocks may also occur in an order different from that noted in the drawings. For example, two successive blocks may actually be executed substantially in parallel, and they may sometimes be executed in the reverse order, depending on the functionality involved. It is also noted that each block in the block diagrams and/or flowcharts, and combinations of blocks in the block diagrams and/or flowcharts may be implemented with a dedicated hardware-based system that performs the specified functions or actions, or may be implemented with a combination of dedicated hardware and computer instructions.

Various embodiments of the present disclosure may be combined with each other without departing from the logic. The description of the various embodiments is focused differently, and reference may be made to the description of other embodiments for parts not described in detail.

Though having described the various embodiments of the present disclosure, the foregoing description is illustrative, not exhaustive, and is not limited to the various embodiments disclosed. Many modifications and variations will be apparent to those of ordinary skill in the art without departing from the scope and spirit of the illustrated embodiments. The choice of terms used herein is intended to best explain the principles of the various embodiments, practical applications, or technical improvements to the technology in the market, or to enable others of ordinary skill in the art to understand the various embodiments disclosed herein.

### INDUSTRIAL APPLICABILITY

In the embodiments, at least one to-be-analyzed object is displayed in response to a first operation for a target object; an anchor point for determining one of the at least one to-be-analyzed object is obtained in response to a second operation for the target object; according to acquired object distribution images and the anchor point, a range of area where the current to-be-analyzed object corresponding to the anchor point is located in the target object is determined. Thus, various positional relationships such as the target object and the anchor point can be clearly obtained in the visual interface design, and the display effect of the interface design is intuitive, so that the user can obtain accurate judgment results based on the intuitive interface design.

1. A method for displaying a target object, comprising:
displaying at least one to-be-analyzed object in response to a first operation for the target object;
obtaining an anchor point for determining one of the at least one to-be-analyzed object, in response to a second operation for the target object; and
determining, according to acquired object distribution images and the anchor point, a range of area where a current to-be-analyzed object corresponding to the anchor point is located in the target object.

2. The method of claim 1, after determining the range of area where the to-be-analyzed object corresponding to the anchor point is located in the target object, the method further comprising:
displaying a feature object that corresponds to the current to-be-analyzed object corresponding to the anchor point in response to a third operation for the current to-be-analyzed object corresponding to the anchor point, wherein the feature object has a nature of lesion different from that of the current to-be-analyzed object corresponding to the anchor point.

3. The method of claim 1, wherein the object distribution images comprise: an image of a distribution range of the at least one to-be-analyzed object in the target object.

4. The method of claim 3, wherein determining, according to the acquired object distribution images and the anchor point, the range of area where the current to-be-analyzed object corresponding to the anchor point is located in the target object comprises:
obtaining, from the object distribution images, a reference image corresponding to the anchor point; and
determining, according to a serial number or ranking position of the reference image among the object distribution images, the range of area where the current to-be-analyzed object corresponding to the anchor point is located in the target object.

5. The method of claim 4, after obtaining, from the object distribution images, the reference image corresponding to the anchor point, the method further comprising:
displaying the reference image corresponding to the anchor point in a display mode different from a mode of displaying a non-reference image among the object distribution images, to distinguish the reference image from the non-reference image, and
feeding an obtained display result back to a user in real time.

6. The method of claim 1, in response to a position change of the anchor point, the method further comprises:
updating a range of area where a position-changed to-be-analyzed object is located in the target object, to obtain an updated result by switching the current to-be-analyzed object to the position-changed to-be-analyzed object and synchronizing the position change of the anchor point to the object distribution images.

7. The method of claim 6, before displaying the at least one to-be-analyzed object in response to the first operation for the target object, the method further comprising:
obtaining a feature vector corresponding to the at least one to-be-analyzed object;
recognizing each of the at least one to-be-analyzed object according to the feature vector and a recognition network; and
identifying each of the at least one to-be-analyzed object to obtain a display identifier, wherein each of the at least one to-be-analyzed object is displayed according to the display identifier.

8. An electronic device, comprising:
a processor; and
a memory configured to store processor-executable instructions, wherein the processor is configured to perform following operations:
displaying at least one to-be-analyzed object in response to a first operation for a target object;
obtaining an anchor point for determining one of the at least one to-be-analyzed object, in response to a second operation for the target object; and
determining, according to acquired object distribution images and the anchor point, a range of area where a current to-be-analyzed object corresponding to the anchor point is located in the target object.

9. The electronic device of claim 8, wherein the processor is further configured to perform following operation:
displaying a feature object that corresponds to the current to-be-analyzed object corresponding to the anchor point in response to a third operation for the current to-be-analyzed object corresponding to the anchor point, wherein the feature object has a nature of lesion different from that of the current to-be-analyzed object corresponding to the anchor point.

10. The electronic device of claim 8, wherein the object distribution images comprise: an image of a distribution range of the at least one to-be-analyzed object in the target object.

11. The electronic device of claim 10, wherein the processor is further configured to perform following operations:
obtaining, from the object distribution images, a reference image corresponding to the anchor point; and
determining, according to a serial number or ranking position of the reference image among the object distribution images, the range of area where the current to-be-analyzed object corresponding to the anchor point is located in the target object.

12. The electronic device of claim 11, wherein the processor is further configured to perform following operations:
displaying the reference image corresponding to the anchor point in a display mode different from a mode of displaying a non-reference image among the object distribution images, to distinguish the reference image from the non-reference image; and
feeding an obtained display result back to a user in real time.

13. The electronic device of claim 8, wherein the processor is further configured to perform following operation:
in response to a position change of the anchor point, updating a range of area where a position-changed to-be-analyzed object is located in the target object to obtain an updated result by switching the current to-be-analyzed object to the position-changed to-be-analyzed object and synchronizing the position change of the anchor point to the object distribution images.

14. The electronic device of claim 13, wherein the processor is further configured to perform following operations:
obtaining a feature vector corresponding to the at least one to-be-analyzed object;
recognizing each of the at least one to-be-analyzed object according to the feature vector and a recognition network; and
identifying each of the at least one to-be-analyzed object to obtain a display identifier, wherein each of the at least one to-be-analyzed object is displayed according to the display identifier.

15. A non-transitory computer-readable storage medium having stored thereon computer program instructions that, when executed by a processor, implement a method for displaying a target object, the comprising:
displaying at least one to-be-analyzed object in response to a first operation for the target object;
obtaining an anchor point for determining one of the at least one to-be-analyzed object, in response to a second operation for the target object; and
determining, according to acquired object distribution images and the anchor point, a range of area where a current to-be-analyzed object corresponding to the anchor point is located in the target object.

16. The non-transitory computer-readable storage medium of claim 15, after determining the range of area where the to-be-analyzed object corresponding to the anchor point is located in the target object, the method further comprising:
displaying a feature object that corresponds to the current to-be-analyzed object corresponding to the anchor point in response to a third operation for the current to-be-analyzed object corresponding to the anchor point, wherein the feature object has a nature of lesion different from that of the current to-be-analyzed object corresponding to the anchor point.

17. The non-transitory computer-readable storage medium of claim 15, wherein the object distribution images comprise: an image of a distribution range of the at least one to-be-analyzed object in the target object.

18. The non-transitory computer-readable storage medium of claim 17, wherein determining, according to the acquired object distribution images and the anchor point, the range of area where the current to-be-analyzed object corresponding to the anchor point is located in the target object comprises:
obtaining, from the object distribution images, a reference image corresponding to the anchor point; and
determining, according to a serial number or ranking position of the reference image among the object distribution images, the range of area where the current to-be-analyzed object corresponding to the anchor point is located in the target object.

19. The non-transitory computer-readable storage medium of claim 18, after obtaining, from the object distribution images, the reference image corresponding to the anchor point, the method further comprising:
displaying the reference image corresponding to the anchor point in a display mode different from a mode of displaying a non-reference image among the object distribution images, to distinguish the reference image from the non-reference image, and
feeding an obtained display result back to a user in real time.

20. The non-transitory computer-readable storage medium of claim 16, in response to a position change of the anchor point, the method further comprises:
updating a range of area where a position-changed to-be-analyzed object is located in the target object, to obtain an updated result by switching the current to-be-analyzed object to the position-changed to-be-analyzed object and synchronizing the position change of the anchor point to the object distribution images.
